(19) United States
(12) Patent Application Publication
(10) Pub. No.: US 2026/0004915 A1
(43) Pub. Date: Jan. 1, 2026

(54) Computer Implemented System and Method for Comprehensive Personal Growth and Well-Being

(71) Applicant: Ann Amaka Sublett, Katy, TX (US)

(72) Inventor: Ann Amaka Sublett, Katy, TX (US)

(21) Appl. No.: 18/756,005

(22) Filed: Jun. 27, 2024

(51) Int. Cl. G16H 20/90

(52) U.S. Cl. CPC G16H 20/90

(57) ABSTRACT

A system and method for supporting personal growth and well-being, comprising a mobile application with a user interface providing access to a plurality of evidence-based tools, including gratitude affirmations, manifestation practices, guided meditations, therapeutic hypnosis, breathwork exercises, and sound therapies. The system receives user input selecting one or more tools, stores user progress data, and displays insights derived from the data. Personalized features include a printable electronic gratitude journal, daily Bible verses, and motivational push notifications based on user goals and app interaction data. An artificial intelligence component analyzes user data to generate personalized recommendations for optimizing the user's personal growth journey. A social community feature enables users to connect, share experiences, and participate in group challenges and accountability programs. The invention provides a comprehensive, personalized, and accessible approach to self-improvement using advanced technologies such as biometric feedback.

### BACKGROUND

### Field of the Invention

The present invention relates generally to the field of personal growth and self-improvement, and more specifically to a comprehensive mobile application that integrates various techniques such as gratitude affirmations, manifestation practices, guided meditations, therapeutic hypnosis, breathwork exercises, and sound therapies to help users transform their lives and achieve their full potential.

### Description of Related Art

In recent years, there has been a growing interest in personal development and self-improvement tools, particularly in the form of mobile applications. These apps often focus on specific techniques such as meditation, goal-setting, or positive affirmations. However, few apps offer a comprehensive, integrated approach that combines multiple evidence-based practices to support users' holistic well-being and personal growth.

Existing personal growth systems, such as the one described in US Patent Application Publication No. US20120084180A1, titled “Personal Growth System, Methods, and Products,” provide web-enabled platforms that offer guided experiences and track users' progress. However, these systems may lack the convenience and accessibility of a mobile application, as well as the breadth of features and personalization capabilities offered by the present invention.

The present invention addresses these limitations by providing a user-friendly, AI-driven platform that integrates a wide range of personal growth techniques, including gratitude affirmations, manifestation practices, guided meditations, therapeutic hypnosis, breathwork exercises, and sound therapies. By offering personalized content recommendations based on users' preferences and goals, as well as a printable electronic gratitude journal and daily motivational notifications, the app aims to provide a more engaging and effective personal development experience compared to existing solutions.

In summary, the present invention represents a significant improvement over existing personal growth systems and applications by offering a comprehensive, evidence-based, and highly personalized approach to self-improvement and holistic well-being in a convenient and accessible mobile format.

### SUMMARY

The present invention is directed to a comprehensive personal growth and well-being system, implemented as a mobile application, that integrates a variety of evidence-based techniques to support users in achieving their full potential. The system includes a user interface that provides access to a plurality of personal growth tools, such as gratitude affirmations, manifestation practices, guided meditations, therapeutic hypnosis sessions, breathwork exercises, and sound therapies.

The mobile application receives user input selecting one or more of the personal growth tools and provides the user with access to the selected tools. User progress data associated with the selected tools is stored in memory, and user progress insights derived from the data are displayed on the user interface.

The invention further includes personalized features such as a printable electronic gratitude journal, a series of daily Bible verses for spiritual guidance, and daily motivational push notifications containing content based on user goals, progress, and app interaction data.

The system leverages artificial intelligence to analyze user data and generate personalized recommendations for optimizing the user's personal growth journey. The app also incorporates a social community feature, enabling users to connect with like-minded individuals, share experiences, and participate in group challenges and accountability programs.

The present invention distinguishes itself from prior art by offering a mobile-first, comprehensive, and highly personalized approach to self-improvement. By integrating multiple evidence-based techniques and leveraging advanced technologies such as biometric feedback, the present invention aims to provide a more engaging, effective, and accessible personal development experience compared to existing solutions.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a block diagram illustrating an embodiment of the hardware and software components of a system for enhancing personal growth and well-being.

FIG. 2 is a flow diagram depicting the process and system components involved in facilitating personal transformation and goal achievement through the mobile application.

FIG. 3 depicts two exemplary user interface screens (301, 302) from a mobile application.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

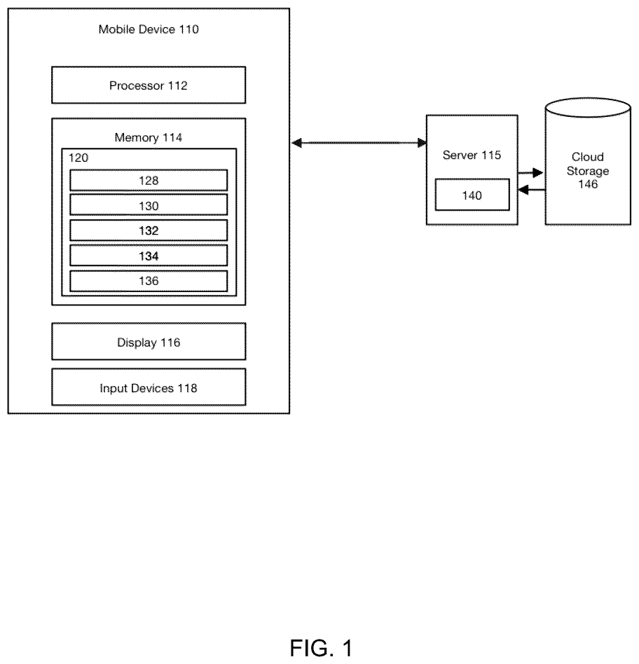

FIG. 1 is a block diagram illustrating an embodiment of the hardware and software components of a system 100 for enhancing personal growth and well-being. The system 100 includes a mobile device 110, such as a smartphone or tablet, which has a processor 112, memory 114, display 116, and input devices 118. The processor 112 can be an ARM-based system-on-a-chip such as Qualcomm Snapdragon, providing high performance and energy efficiency. The memory 114 includes both volatile RAM for temporary storage and non-volatile flash memory for persistent storage. Input devices 118 can include touchscreens, keyboards, microphones for voice input, and sensors such as accelerometers and gyroscopes. The system 100 also includes a server 115 that hosts backend services and databases. The server 115 has a multi-core x86-64 processor, such as Intel Xeon, and high-capacity RAM and solid-state drives for storage. In some embodiments input devices 118 can include laptops, mobile phones, tablets or any other personal computing devices that communicate input information to the mobile device 110 and server 115 over wireless networks such as Wi-Fi or cellular data connections.

The memory 114 stores a mobile application 120 that provides a user interface and implements the logic for the personal growth tools. The mobile application 120 is built using the Flutter framework and written in the Dart programming language. Flutter enables cross-platform development, allowing the app to run on both iOS and Android devices while sharing a single codebase. Dart provides the core logic, business rules, state management, and user interface rendering capabilities. The Flutter framework leverages the Skia graphics library for high-performance rendering and the Dart VM for fast execution. The mobile application 120 uses SQLite for local data persistence and JSON for data serialization.

The mobile application 120 leverages several components of the Firebase platform 140, which runs on the server 115, for backend services and data storage. Firebase Authentication handles user registration, login, and account management using industry-standard security protocols such as OAuth 2.0 and JSON Web Tokens (JWT). Cloud Firestore, a NoSQL database built on Google's infrastructure, stores user data, preferences, and progress information. It provides real-time synchronization, offline support, and automatic scaling. Cloud Storage 146 securely stores user-generated content like journal entries, audio recordings, and images using Google Cloud Storage infrastructure. Firebase Cloud Messaging enables push notifications for delivering personalized content and reminders using the Apple Push Notification Service (APNS) and Google Cloud Messaging (GCM) protocols. Firebase Analytics provides insights into user engagement and behavior using machine learning and big data processing techniques.

In some embodiments the mobile application 120 incorporates various modules designed to facilitate personal growth. The Gratitude module 122 provides personalized gratitude affirmations at predetermined intervals, enables users to document reflections in a digital journal, and offers basic functionalities to share their progress on social media using APIs such as Facebook Graph API and Twitter API. The Manifestation module 124 features guided visualization exercises and goal-setting prompts, tailoring its content based on the user's progress data stored in Cloud Firestore. The Meditation module 126 presents guided meditations, which are organized by duration, purpose, and user experience level, and allows users to choose their preferred background music and nature sounds. The audio files are stored in Cloud Storage 146 and streamed using the ExoPlayer library. In the Hypnosis module 128, users can participate in therapeutic sessions led by certified hypnotherapists, targeting specific issues such as stress, anxiety, and habits, with progress monitored through user feedback stored in Cloud Firestore. The Breathwork module 130 offers visual and auditory guidance and real-time biometric feedback for controlled breathing exercises, with capabilities to sync with wearable devices using Bluetooth Low Energy (BLE) protocol. The Sound Therapy module 132 employs research-supported frequencies, binaural beats, isochronic tones, and solfeggio sequences to enhance cognitive and emotional states. The audio processing is done using the Flutter Sound library. The Spirituality module 134 provides daily Bible verses for spiritual guidance and inspiration, featuring notification functionality using Firebase Cloud Messaging and calendar integration using the device's native calendar APIs. Lastly, the Motivation module 136 sends personalized push notifications containing motivational content that aligns with the user's goals and interactions with the app, using machine learning algorithms running on the server 115.

The mobile application 120 communicates with the server 115 using secure HTTPS connections with SSL/TLS encryption. The server 115 runs a Node.js runtime environment and uses the Express.js framework for building RESTful APIs that handle requests from the mobile application 120. The server 115 also runs background jobs and scheduled tasks using tools like Cron and Celery.

In summary, the system 100 combines a feature-rich mobile application 120 built with Flutter and Dart, with the powerful backend capabilities of Firebase 140 running on the server 115. The mobile application modules (122-136) work together to provide a comprehensive set of personal growth tools. User data is securely stored and synchronized in real-time across devices using Cloud Firestore and Cloud Storage. The server 115 handles compute-intensive tasks and background processing. The Apple App Store and Google Play Store enable broad distribution to users worldwide. This architecture allows the system to deliver an engaging, data-driven, and personalized experience to support users' personal growth and well-being journeys.

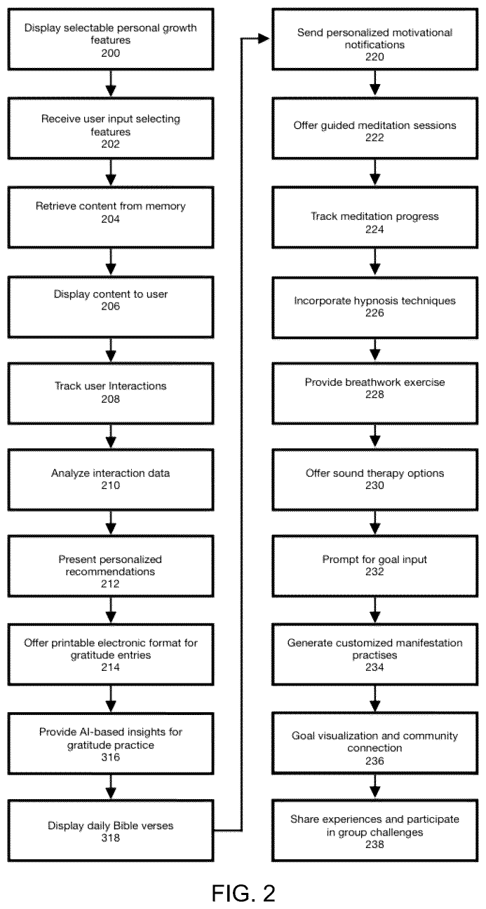

FIG. 2 is a flow diagram depicting an embodiment of the process and system components involved in facilitating personal transformation and goal achievement through the mobile application 120. The process begins with the mobile application 120 displaying a user interface on the display 116 of the mobile device 110, presenting a plurality of selectable personal growth features (step 200). These features include a gratitude module 122 for recording daily reflections and gratitude entries, a spirituality module 134 containing a database of daily Bible verses offering spiritual guidance and inspiration, and a motivation module 136 for delivering personalized motivational push notifications to inspire and uplift the user.

Upon receiving user input via the input devices 118, indicating a selection of one or more personal growth features (step 202), the processor 112 retrieves the associated content from the memory 114 (step 204). The retrieved content is then displayed to the user through the mobile application's user interface on the display 116 (step 206).

As the user interacts with the selected personal growth features, the mobile application 120 tracks these interactions and stores the associated user interaction data in the memory 114 (step 208). The processor 112 then analyzes the user interaction data (step 210) to generate personalized recommendations for optimizing the user's personal growth journey. These recommendations are presented to the user via the mobile application's user interface on the display 116 (step 212).

The gratitude module 122 includes additional features such as a printable electronic format, allowing users to download and print physical copies of their reflections and gratitude entries (step 214). Moreover, the gratitude module 122 incorporates an AI-based analysis feature that processes user entries to provide personalized insights and recommendations for enhancing the user's gratitude practice (step 216).

The spirituality module 134 comprises a database of 366 unique daily Bible verses, each associated with a specific calendar date. This allows users to access a new spiritually uplifting verse each day of the year (step 218).

The motivation module 136 utilizes an AI-driven personalization system that analyzes user interaction data, personal preferences, and behavioral patterns using machine learning algorithms, such as collaborative filtering and content-based filtering, to deliver highly relevant and impactful motivational content through daily push notifications (step 220). The push notifications are sent using the Firebase Cloud Messaging service.

The mobile application 120 further includes a meditation module 126, which offers a library of guided meditation sessions designed to address specific user needs, such as stress reduction, focus enhancement, and emotional well-being (step 222). The meditation module 126 also incorporates a progress tracking system that monitors user engagement with meditation sessions and provides visual feedback on the user's meditation habits and consistency (step 224).

Additionally, the meditation module 126 incorporates therapeutic hypnosis techniques, such as positive suggestion and visualization, through a hypnosis module 128 to facilitate deeper relaxation and personal transformation (step 226).

The mobile application 120 also features a breathwork module 130, which provides a collection of breathwork exercises that guide users through controlled breathing techniques designed to promote relaxation, reduce stress, and improve overall well-being (step 228).

A sound therapy module 132 is included in the mobile application 120, offering a variety of healing frequencies and soothing soundscapes, each tailored to specific user needs, such as improved sleep, reduced anxiety, and enhanced focus (step 230).

The manifestation module 124 offers a personalized goal-setting and manifestation system that prompts users to input their personal goals and aspirations (step 232), generates customized manifestation practices based on user input, and provides a structured approach to visualizing and achieving goals through regular practice and reflection (step 236).

Lastly, the mobile application 120 includes a social community feature that enables users to connect with like-minded individuals pursuing personal growth, share experiences, insights, and support within the app's secure community environment, and participate in group challenges and accountability programs to foster motivation and consistency in personal development practices (step 238).

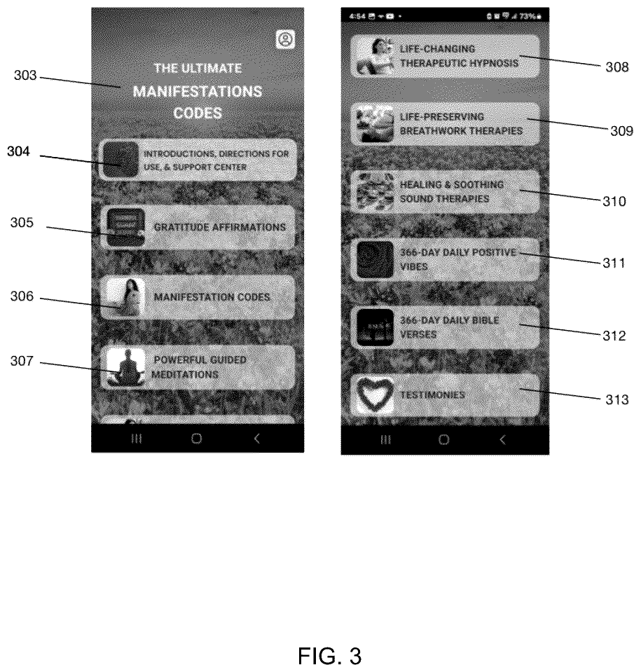

FIG. 3 depicts two exemplary embodiments of user interface screens (301, 302) from a mobile application titled “The Ultimate Manifestations Codes.” Each screen (301, 302) displays a series of menu options overlaid on a background image of a lavender field at sunset illustrating the comprehensive nature of the application.

The left screen (301) represents the top portion of the application's home page. At the top of screen (301), a header (303) prominently displays the application title “THE ULTIMATE MANIFESTATIONS CODES.” Below the header (303), several menu options are presented, each accompanied by a representative icon. The menu options on screen (301) include “Introductions, Directions for Use, & Support Center” (304) depicted with an icon of a black rock, “Gratitude Affirmations” (305) accompanied by an icon of a sign that reads “THANKFUL & GRATEFUL,” “Manifestation Codes” (306) shown with an image of a woman sitting and reading, and “Powerful Guided Meditations” (307) represented by an icon of a meditating figure.

The right screen (302) represents the view when a user scrolls down from the top of the home page. Screen (302) presents additional menu options, including “Life-Changing Therapeutic Hypnosis” (308) illustrated with an image of a woman receiving hypnosis, “Life-Preserving Breathwork Therapies” (309) shown with an image of a group practicing breathwork, “Healing & Soothing Sound Therapies” (310) depicted with an image of sound therapy instruments, “366-Day Daily Positive Vibes” (311) represented by an image of a rose, “366-Day Daily Bible Verses” (312) accompanied by an image of a book with the word “JESUS,” and “Testimonies” (313) depicted with an image of a heart made of red ribbon.

The menu options (304-313) serve as interactive elements, allowing the user to navigate to different sections of the application. When a user selects a menu option (304-313), the application directs the user to a corresponding screen that facilitates the related activity or provides access to the associated resources.

The background image and consistent design elements used across both screens (301, 302) enhance the visual coherence and aesthetic appeal of the application's user interface. The clear labeling of the header (303) and menu options (304-313) ensures ease of navigation and understanding of the available features within the application.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A system for enhancing personal growth and well-being, comprising:
a processor;
a memory coupled to the processor, the memory storing instructions that, when executed by the processor, cause the system to:
display a user interface on a mobile device, the user interface providing access to a plurality of personal growth tools, including:
gratitude affirmations configured to promote a positive mindset, manifestation practices providing structured techniques for achieving goals, guided meditations offering step-by-step instructions for meditation, therapeutic hypnosis sessions addressing specific user issues, breathwork exercises teaching controlled breathing techniques,
sound therapies utilizing healing frequencies and soothing sounds;
receive user input selecting one or more of the personal growth tools;
provide the user with access to the selected personal growth tools;
store user progress data associated with the selected personal growth tools in the memory; and
display user progress insights derived from the user progress data on the user interface.

2. The system of claim 1, wherein the gratitude affirmations are personalized based on user preferences and inputs, and are displayed on the user interface at scheduled intervals throughout the day.

3. The system of claim 1, wherein the manifestation practices comprise a series of guided visualization exercises and goal-setting prompts that adapt based on user progress data stored in the memory.

4. The system of claim 1, wherein the guided meditations are categorized by duration, purpose, and experience level, and include optional background music and nature sounds selectable by the user.

5. The system of claim 1, wherein the therapeutic hypnosis sessions are conducted by certified hypnotherapists and target specific user issues such as stress, anxiety, sleep disorders, and habit control, with progress tracked via user feedback.

6. The system of claim 1, wherein the breathwork exercises include visual guidance, auditory cues, and real-time biometric feedback collected from wearable devices paired with the mobile app.

7. The system of claim 1, wherein the sound therapies utilize research-backed frequencies, binaural beats, isochronic tones, and solfeggio sequences to promote specific cognitive and emotional states, such as focus, relaxation, and creativity.

8. The system of claim 1, further comprising a printable electronic gratitude journal feature that allows users to record daily reflections, affirmations, and manifestation progress, with data exportable and shareable across devices.

9. The system of claim 1, further comprising a series of daily Bible verses curated to provide spiritual guidance and inspiration, with optional notifications and synchronization with the user's calendar.

10. The system of claim 1, further comprising a series of daily motivational push notifications delivered at user-specified times, containing personalized content based on user goals, progress, and app interaction data stored in the memory.

11. A computer-implemented method for facilitating personal transformation and goal achievement, the method comprising:
providing a mobile application user interface, by a processor, the user interface displaying a plurality of selectable personal growth features, including:
a gratitude journal for recording daily reflections and gratitude entries, a database of daily Bible verses,
daily motivational push notifications delivering personalized messages;
receiving, by the processor, user input indicating a selection of one or more of the personal growth features;
retrieving, from a memory, content associated with the selected personal growth features;
displaying the retrieved content to the user via the mobile application user interface;
tracking user interactions with the selected personal growth features and storing associated user interaction data in the memory;
analyzing the user interaction data, by the processor, to generate personalized recommendations for optimizing the user's personal growth journey; and
presenting the personalized recommendations to the user via the mobile application user interface.

12. The method of claim 11, wherein the gratitude journal further comprises:
a printable electronic format for users to download and print physical copies of their reflections and gratitude entries; and
an AI-based analysis feature that processes user entries to provide personalized insights and recommendations for enhancing the user's gratitude practice.

13. The method of claim 11, wherein the database of daily Bible verses are organized into 366 unique verses, each associated with a specific calendar date.

14. The method of claim 11, wherein the daily motivational push notifications are generated using an AI-driven personalization system that analyzes user interaction data, personal preferences, and behavioral patterns to deliver highly relevant and impactful motivational content.

15. The method of claim 11, further comprising:
a library of guided meditation sessions designed to address specific user needs, such as stress reduction, focus enhancement, and emotional well-being; and
a progress tracking system that monitors user engagement with meditation sessions and provides visual feedback on the user's meditation habits and consistency.

16. The method of claim 17, wherein the guided meditation sessions incorporate therapeutic hypnosis techniques, such as positive suggestion and visualization, to facilitate deeper relaxation and personal transformation.

17. The method of claim 11, further comprising a collection of breathwork exercises that guide users through controlled breathing techniques designed to promote relaxation, reduce stress, and improve overall well-being.

18. The method of claim 11, further comprising a sound therapy module that offers a variety of healing frequencies and soothing soundscapes, each tailored to specific user needs comprising improved sleep, reduced anxiety, and enhanced focus.

19. The method of claim 11, further comprising a personalized goal-setting and manifestation system that:
prompts users to input their personal goals and aspirations;
generates customized manifestation practices based on user input; and
provides a structured approach to visualizing and achieving goals through regular practice and reflection.

20. The method of claim 11, further comprising a social community feature that enables users to:
connect with like-minded individuals pursuing personal growth;
share experiences, insights, and support within the app's secure community environment; and
participate in group challenges and accountability programs to foster motivation and consistency in personal development practices.
